(19) United States
(12) Patent Application Publication
(10) Pub. No.: US 2022/0079983 A1
(43) Pub. Date: Mar. 17, 2022

(54) NANOPARTICLES AND PREPARATION METHOD

(71) Applicants: Centre National de la Recherche Scientifique, Paris (FR); Université Paris-Saclay, Gif Sur Yvette (FR)

(72) Inventors: Daniela Edith SALADO LEZA, San Luis Potosi (MX); Hynd REMITA, Orsay (FR); Erika PORCEL, Palaiseau (FR); Sandrine LACOMBE, Saint-Cyr-Sous-Dourdan (FR)

(21) Appl. No.: 17/420,507

(22) PCT Filed: Jan. 2, 2020
(86) PCT No.: PCT/EP2020/050045

§ 371 Date: Jul. 2, 2021

(30) Foreign Application Priority Data
Jan. 2, 2019 (FR) 1900008

(51) Int. Cl. A61K 33/245; A61K 9/14; A61K 47/69; A61K 47/60; A61K 33/243; A61K 33/242; A61P 35/00

(52) U.S. Cl. CPC A61K 33/245; A61K 9/14; A61K 47/6929; A61P 35/00; A61K 33/243; A61K 33/242; A61K 47/60

(57) ABSTRACT

The present invention relates to nanoparticles of a metal chosen from among platinum, bismuth or a mixture thereof, which are functionalised at their surface by functionalised polyethylene glycol, comprising in particular at least one OH, COOH, NH₂ or SH functional group, and to their method of preparation, which comprises of the following steps:
a) Mixing a precursor of nanoparticles with a functionalised polyethylene glycol, in particular comprising at least one OH, COOH, NH₂ or SH functional group, in water;
b) Exposing the mixture to ionising radiation.

The present invention relates to nanoparticles of platinum, gold and bismuth, which may be used in particular in the field of diagnostics and therapy, and a nanoparticle preparation method for preparing such types of nanoparticles.

Nanoparticles are generally synthesized via chemical processes that include a number of steps. Consequently, these methods of synthesis have yields of less than 100% and are often difficult to reproduce. Moreover, the majority of these methods require the use of chemical reducing agents and solvents that are toxic to greater or lesser degrees. It is therefore necessary to provide for rinsing and sterilization steps whereby it is possible to render these particles biocompatible and non-toxic, such that they may therefore be able to be injected into humans. These additional steps of rinsing and sterilization thus prolong the preparation time for preparing the nanoparticles and reduce the yields thereof.

It is therefore of significant interest to provide a nanoparticle preparation method for effectively preparing nanoparticles with a high yield which also makes it possible to obtain particles that are non-toxic and biocompatible, all in one single step.

One objective of the present invention is to provide a nanoparticle preparation method for preparing nanoparticles, the method producing a high yield and with a possibility of scale-up.

Another objective of the present invention is to provide such a method that makes it possible to obtain particles that are non-toxic and biocompatible, in one single step.

Yet another objective of the present invention is to provide non-toxic, biocompatible and sterile nanoparticles which can in particular be used in diagnostics and in ionising radiation therapy (radiation therapy, hadron therapy, proton therapy, curie therapy—also known as radium therapy).

Still other objectives will become apparent upon reading the description of the invention which follows.

The present invention relates to nanoparticles of a metal chosen from among platinum, gold or bismuth, or a mixture thereof, which are functionalised at their surface with functionalised polyethylene glycol.

The present invention relates to nanoparticles of a metal chosen from among platinum, gold or bismuth, which are functionalised at their surface with functionalised polyethylene glycol.

In the context of the present invention, the term “functionalised polyethylene glycol” is understood to refer to a polyethylene glycol comprising at least one functional group that serves to enable interaction or ligandation with the surface of platinum, gold or bismuth, for example these groups are OH, NH₂, SH, COOH functional groups.

Preferably, the nanoparticles have a mean diameter of between 1 nm and 100 nm, preferably between 1 nm and 10 nm. The mean diameter of the nanoparticles is obtained by performing measurement using transmission electron microscopy (TEM).

Preferably, the nanoparticles have a mean hydrodynamic diameter of between 5 and 100 nm, preferably between 10 and 20 nm. In the context of the present invention, the term “hydrodynamic diameter” is understood to refer to the diameter as measured by the Dynamic Light Scattering (DLS) technique.

Preferably, the nanoparticles of the invention have a mean zeta potential of between −5 and −30 mV, preferably between −10 and −20 mV. The mean zeta potential is preferably measured by zetametry.

In a particularly preferred and advantageous manner, the nanoparticles of the invention do not exhibit any trace of solvents other than water.

The present invention also relates to an aqueous solution comprising the nanoparticles according to the invention described here above.

The present invention also relates to a nanoparticle preparation method for preparing nanoparticles of a metal chosen from among platinum, gold or bismuth, or one of the mixtures thereof, in particular the nanoparticles of the invention described here above, the method comprising of the following steps:
a) Mixing a precursor of nanoparticles with a functionalised polyethylene glycol in water;
b) Exposing the mixture to ionising radiation: gamma radiation, X-rays, accelerated electrons or accelerated ions.

The present invention also relates to a nanoparticle preparation method for preparing nanoparticles of a metal chosen from among platinum, gold or bismuth, in particular the nanoparticles of the invention described here above, the method comprising of the following steps:
a) Mixing a precursor of nanoparticles with a functionalised polyethylene glycol in water;
b) Exposing the mixture to ionising radiation: gamma radiation, X-rays, accelerated electrons or accelerated ions.

Preferably the metal is platinum, or a mixture of platinum and bismuth.

In the context of the present invention, the term “precursor of nanoparticles” is understood to refer to any compound known to the person skilled in the art that serves to enable the preparation of nanoparticles. In particular, the precursors are chosen from among platinum salts (in particular tetraamine platinum(II) chloride (Pt(NH₃)₄Cl₂, H₂O), chloroplatinic acid (H₂PtCl₆), potassium tetrachloroplatinate (K₂PtCl₄)), gold salts (in particular chloroauric acid (HAuCl₄)), bismuth salts (in particular bismuth chloride (BiCl₃), bismuth nitrate (Bi(NO₃)₃), and oxides (in particular bismuth oxide (Bi₂O₃))

After the step b), the particles may be lyophilised in particular in order to be stored, it being possible subsequently to cause the particles to be resuspended, for example in water, prior to use.

Preferably, the mixture is degassed under an inert gas, for example nitrogen or argon, prior to exposure to ionising radiation.

Preferably, the solution is irradiated with ionising radiation, preferably with energy values comprised between 5 keV and 5 MeV, such as gamma radiation deriving from a source of Cobalt 60, X-rays, beams of accelerated electrons or accelerated ions, in particular medical radiation of carbon ions as used in hadrontherapy.

In a particularly advantageous manner, the doses used for the reduction of platinum, gold or bismuth depend on the precursor, on the initial concentration of precursor and on the source of irradiation: they vary from 1 to 20 kGy for concentrations of metal ranging from 0.5 to 20 mM, for example the dose is typically 10 kGy in order to completely reduce 10⁻³ mol/L of metal complex containing a salt that comprises Pt²⁺. The radiation exposure step of exposing to gamma radiation thus extends over a period of between 1 and 20 hours, at a dose rate of 95.5 Gy·min⁻¹.

The functionalised polyethylenes used in the method of the invention are, for example, Hydroxyl poly(ethylene glycol) [PEG-OH], α-hydroxyl-ω-carboxyl poly(ethylene glycol) [HO-PEG-COOH], α-Amino-ω-mercapto poly(ethylene glycol) [NH₂-PEG-SH], Poly(ethylene glycol) diamine [PEG-2NH₂], and PEG-thiol [PEG-SH]. In an advantageous manner, the use of functionalised polyethylene glycol makes it possible to ensure that the nanoparticles obtained exhibit stability and biocompatibility. Preferably, the polyethylene glycols used in implementation according to the invention have a molar mass MW of between 600 and 3000, preferably between 1000 and 2000 g/mol. In a particularly preferable manner, the polyethylene glycols used in implementation according to the invention are PEG-OH and PEG-2NH₂, preferably having an MW of between 1000 and 2000 g/mol.

In a particularly advantageous manner, the functionalisation with the PEG advantageously makes possible the post-synthesis grafting of labelling molecules, for example fluorophores. Thus, the present invention also relates to nanoparticles of a metal chosen from among platinum, gold or bismuth, or a mixture thereof, which are functionalised at their surface with functionalised polyethylene glycol and post-functionalised with a labelling molecule, for example a fluorophore. The invention also relates to the nanoparticle preparation method for preparing the above-noted nanoparticles which in addition comprises a step of post-functionalisation with a labelling molecule, for example a fluorophore. This labelling, a post-functionalisation step, may be carried out by any method known to the person skilled in the art.

In the method according to the invention, the precursor of nanoparticles is preferably used in implementation in an amount ranging from 10⁻⁴ mol/L to 10⁻² mol/L, preferably from 3×10⁻³ mol/L to 5×10⁻⁴ mol/L, in terms of molar concentration in the mixture of the step a).

In the method according to the invention, the functionalised polyethylene glycol is preferably used in implementation in an amount ranging from 10⁻³ to 5×10⁻¹ mol/L and preferably from 10⁻² to 10⁻¹ mol/L, in terms of molar concentration in the mixture of the step a).

Preferably, in the method of the invention, the molar ratio between the precursor of nanoparticles and the functionalised polyethylene glycol is between 10 and 1000, preferably between 25 and 100.

In a particularly preferred and advantageous manner, the method of the invention is effectively implemented with water being the sole solvent therein. Thus, in a particularly advantageous manner, the method of the present invention does not make use in implementation of any solvent, in particular any organic solvent, or any toxic solvent. This advantageously makes it possible to obtain nanoparticles or a suspension of nanoparticles in water which are biocompatible and which may be used directly without an intermediate step of washing.

The method of the present invention advantageously makes it possible to obtain ready-to-use nanoparticles in a sterile solution, in one single step. The reducing species are derived from the radiolysis of water (electrons and H radicals), moreover with no chemical reducing agent being added to the solution. It is therefore a “clean” reduction process.

The operational implementation of radiolysis (gamma radiation, accelerated electrons or ions, X-rays), while effectively serving to enable the synthesis of nanoparticles, also kills any living organisms that could be present therein and thereby sterilises the solution obtained.

Moreover, and in a particularly advantageous manner, the method of the present invention makes possible a conversion of 100% of the metal precursor. Since the method only uses water, it is not necessary to carry out various steps that are tedious and time consuming, and costly in terms of product and time—such as washing of the nanoparticles, which prevents loss of the product (as is generally observed when a step of filtration is necessary, which is not the case in this instance).

Thus, the method of the present invention makes it possible to obtain nanoparticles that are homogeneous in size, and to obtain a sterile colloidal solution composed exclusively of nanoparticles of a metal chosen from among platinum, gold or bismuth.

In addition, the method of the invention makes it possible to obtain stable nanoparticles. The term “stable nanoparticles” is understood to refer to nanoparticles that do not undergo any change in size (in particular no alteration of the metallic body of the nanoparticles), nor in degrees of oxidation over a span of several weeks.

The present invention also relates to an aqueous solution of nanoparticles that is possible to obtain by the method described here above.

The present invention also relates to nanoparticles that are capable of being obtained by the method of the invention.

In a particularly advantageous manner, the presence of the biocompatible polymer, functionalised PEG, at the surface of the nanoparticles, as well as their small size promotes the accumulation of the said nanoparticles, for example in tumours, in particular by the EPR (enhanced permeability and retention) effect. The nanoparticles as claimed by the invention, or that are capable of being obtained by the method of the invention, have the property of amplifying the effects of ionising radiation (photon or hadron radiation) such as radiation-induced cell death. Thus, the present invention also relates to nanoparticles as claimed by the invention or that are capable of being obtained by the method of the invention for the use thereof in the treatment of cancers and tumours, in particular for amplifying the effects of medical radiation used for the treatment of these cancers or tumours.

The present invention also relates to a treatment amplification method for amplifying the effects of ionising radiation used for the treatment of a cancer or tumour in a patient, the method comprising the injection into the said patient of an effective amount of the nanoparticles as claimed by the invention or that are capable of being obtained by the method of the invention.

In a particularly advantageous manner, the nanoparticles as claimed by the invention, or that are capable of being obtained by the method of the invention, are composed of metals with a high atomic number and a high electron density, which makes it possible for them to be imaged, in particular by means of the technique referred to as Computed Tomography (CT). Thus, the present invention also relates to nanoparticles as claimed by the invention, or that are capable of being obtained by the method of the invention for the use thereof in medical imaging for diagnostics.

The present invention will now be described with the aid of non-limiting examples.

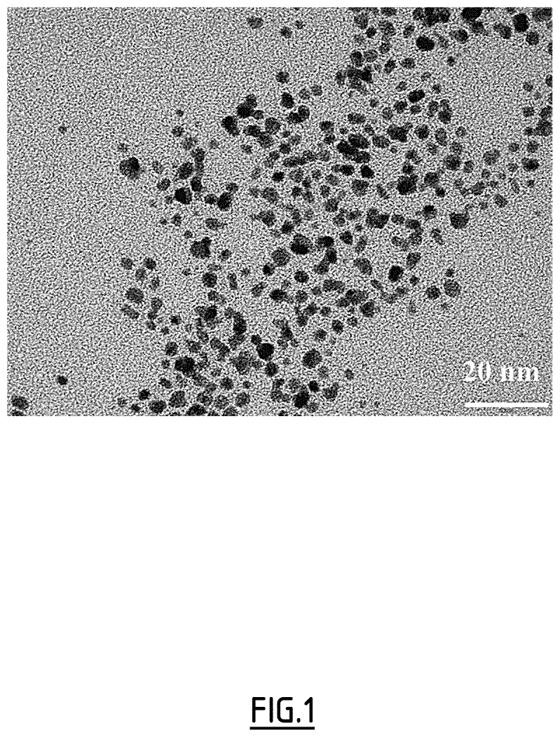

FIG. 1 is an image of the platinum nanoparticles functionalised by PEG-OH as viewed by High Resolution Transmission Electron Microscopy (HRTEM).

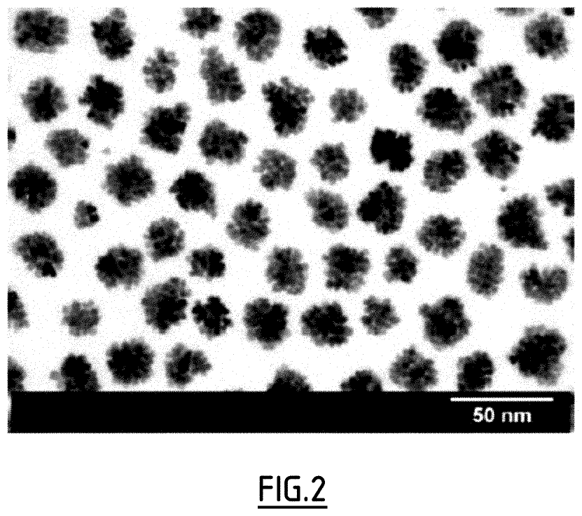

FIG. 2 is an image of the platinum nanoparticles functionalised by PEG-2NH2 as viewed by High Resolution Transmission Electron Microscopy (HRTEM).

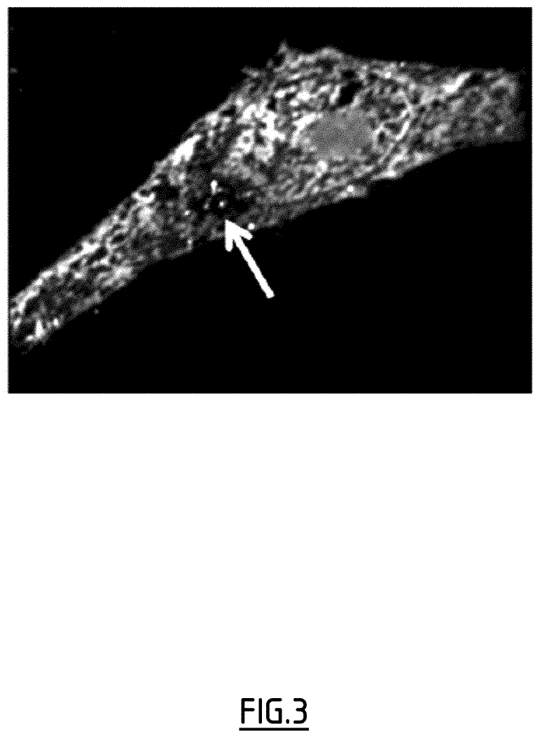

FIG. 3 shows the internalisations of particles by the HeLa cells.

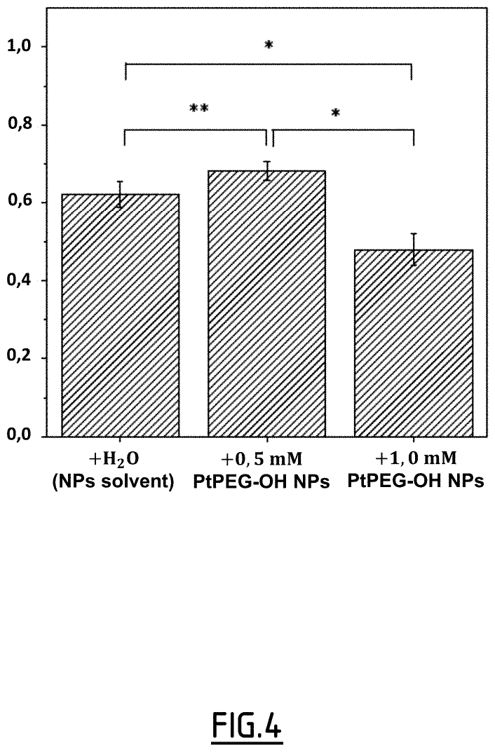

FIG. 4 shows the mitotic survival of HeLa cells in the presence or absence of platinum nanoparticles (PtNPs) (6 hours of incubation at 0.5 mM and 1 mM).

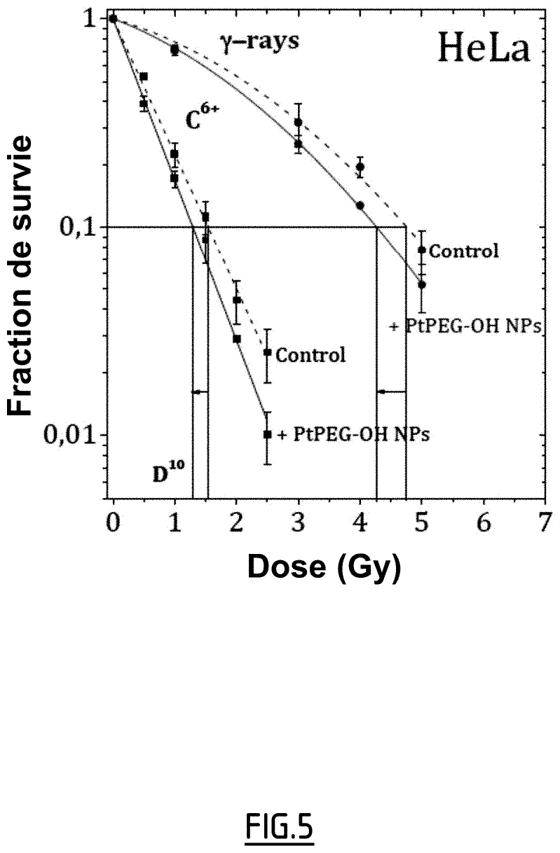

FIG. 5 shows the survival of HeLa cells treated with radiation of C⁶⁺ ions or Cs-137 gamma photons, with and without platinum nanoparticles.

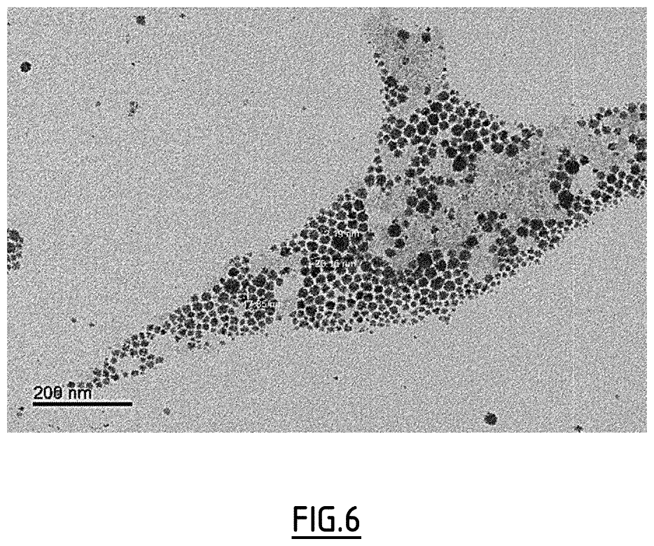

FIG. 6 shows an image of the platinum and bismuth nanoparticles functionalised by PEG-2NH₂ as viewed by High Resolution Transmission Electron Microscopy (HRTEM).

### EXAMPLE 1: PREPARATION OF THE NANOPARTICLES

The platinum nanoparticles (PtNPs) are synthesised by using a platinum precursor, tetraamineplatinum(II) chloride (Pt(NH₃)₄Cl₂, 2H₂O). This precursor is diluted in water in a proportion of 50 mg in 10 mL. A sample of this solution (6.67 mL) is mixed with 2 mL of polyethylene glycol 1000 (PEG-OH) (5 M) or polyethylene diamine 2000 (PEG-2NH₂), the mixture is diluted in water (1.33 mL). This solution is degassed under nitrogen. It is then exposed for a period of approximately 17.5 hours, to radiation deriving from a cobalt 60 gamma source (dose rate: 95.5 Gy/min). This results in a colloidal solution that is black in colour, sterile, and composed exclusively of homogeneous platinum nanoparticles functionalised with PEG (—OH or -2NH₂).

In the case of PEG-OH, the platinum core has a spherical shaped form and with a diameter equal to 3.2 nm (FIG. 1). In the case of PEG-2NH₂, aggregates are obtained in flower-shaped forms, that measure 14.6 nm in size and contain nanoparticles (NPs) measuring 3.2 nm in diameter (FIG. 2).

The hydrodynamic diameter of the nanoparticles is obtained by means of Dynamic Light Scattering (DLS) measurements. It is 8.8 nm in the case of PEG-OH and 16.1 nm in the case of PEG-2NH₂. The platinum nanoparticles functionalised with PEG-OH have a mean zeta potential of −17 mV. Additional measurements by X-ray Photoelectron Spectrometry (XPS) confirmed the absence of platinum precursor in the solution of platinum nanoparticles (PtNPs) and shows that all of the platinum has been reduced in its entirety (zero oxidation number), which proves that this method of synthesis provides a yield of 100%.

The bismuth-platinum nanoparticles (Bi/PtNPs) are synthesised by using a platinum precursor, tetraamineplatinum(II) chloride (Pt(NH₃)₄Cl₂, 2H₂O) and a bismuth precursor, Ammonium Bismuth Citrate (ABC, C₆H₈BiNO₇). The platinum precursor is diluted in water in a proportion of 50 mg in 10 mL, while the bismuth precursor is diluted in water in a proportion of 41.5 mg in 10 mL.

A sample of the platinum solution (1.38 mL) is mixed with a sample of the bismuth solution (0.5 mL), and with 2.5 mL of 100 mM polyethylene diamine 2000 (PEG-2NH₂); thereafter this mixture is diluted in water (1.3 mL). A solution is obtained with a Bi/Pt atomic ratio of 1:3.7 and a Bi/PEG-2NH₂ ratio of 1:50 mol/mol %.

This solution is degassed under nitrogen. It is then exposed for a period of approximately 4 hours to radiation deriving from a cobalt 60 gamma source (dose rate: 37 Gy/min). This results in a colloidal solution that is black in colour, sterile, and composed exclusively of homogeneous Bismuth-Platinum nanoparticles functionalised with PEG-2NH₂. According to the TEM images, the platinum core has a spherical shaped form and a diameter equal to 21.2+/−12.9 nm.

The hydrodynamic diameter of the nanoparticles, which is obtained by means of Dynamic Light Scattering (DLS) measurements, is 35 nm. Additional measurements by X-ray Photoelectron Spectrometry (XPS) confirmed the absence of platinum and bismuth precursor in the solution of nanoparticles, given that it shows that all of the metals have been reduced in their entirety (zero oxidation number), which proves that this method of synthesis provides a yield of 100%.

### Example 2: Implementation and Use of the Nanoparticles

After incubation for a period of 6 hours of the cells (HeLa) with a solution of PtNPs functionalised with PEG-OH containing 0.5 mM of platinum (obtained in Example 1), the latter are internalised by HeLa cells (FIG. 3) at a rate of 1.6 pg per cell, which corresponds to 49×10⁵ PtNPs per cell (quantification carried out by Inductively Coupled Plasma Mass Spectrometry or ICP-MS).

The PtNPs functionalised with PEG-OH or PEG-2NH₂ exhibit a low level of cytotoxicity on the human cell lines. In fact, the HeLa cells incubated for a period of 6 hours with a solution of PtNPs (platinum nanoparticles) containing 0.5 mM or 1 mM of platinum, respectively, exhibit low mitotic death (<10%+/−5%) (FIG. 4).

When the nanoparticles are activated by ionising radiation (gamma radiation from Cobalt 60 or Cesium 137, X-rays, or indeed even medical radiation of carbon ions as used in hadrontherapy), they exhibit the property of amplifying molecular damage and radiation-induced cell death. For example, preliminary studies on nano-bioprobes show that the presence of platinum nanoparticles functionalised with PEG-OH amplifies by a factor of 2 the number of instances of molecular damage of nanometric dimensions induced by ion irradiation.

Studies on human tumour cells (HeLa) show that the presence of platinum nanoparticles functionalised with PEG-OH amplifies the radiotoxicity (cell death) of a medical radiation beam such as carbon ions or gamma photons. In fact, when HeLa cells are incubated for a period of 6 hours with a solution of PtNPs containing 0.5 mM of platinum, they exhibit cell death (as measured by clonogenic survival) induced by irradiation (by accelerated carbon ions or gamma photons from Cs 137), at a greater level than the cell death in the control cells (not comprising NPs) (FIG. 5).

### Example 3: Implementation and Use of the Nanoparticles

In vivo experiments on healthy mice were carried out by injecting intravenously, into the tail of the mouse, 200 μl of PEG-OH functionalised nanoparticles with 10 g/l of Pt obtained according to the protocol of I Example 1, lyophilised and re-suspended in sterile water. A dose of 0.1 g of nanoparticles per kg were injected into the mice.

The tests did not show any toxicity thus confirming the biocompatibility of this product, a slight enhancement of the contrast in computed tomography (CT) was clearly evidenced in the bladder. This enhancement shows that if the nanoparticles get accumulated in the bladder, it signifies that the renal system is capable of excreting the nanoparticles that would not have been internalised by the tumour, and which would thus lead to little to no accumulation in healthy organs.

### Example 4: Functionalisation of the Nanoparticles with Rhodamine

The platinum nanoparticles (PtNPs) are synthesised as described in Example 1 by using a platinum precursor, tetraamineplatinum(II) chloride (Pt(NH₃)₄Cl₂, 2H₂O), and polyethylene glycol diamine 2000 (PEG-2NH₂). Thereafter, these NPs have grafted on to them, a fluorescent marker or label: rhodamine B isothiocyanate or RB ITC (C₂₉H₃₀ClN₃O₃S). Thus, the amine functional group (NH₂) of the PEG reacts with the isothiocyanate of the marker (formation of thiourea). For this, it is necessary to prepare a mixture in water of 4 ml containing these nanoparticles at a concentration of 5 mM of platinum and containing the RB ITC at a concentration of 0.5 mM. This mixture is then agitated at ambient temperature for a period of 24 hours. The labelled nanoparticles that are obtained are then ultrafiltered in water by centrifugation until such time as all the free marker is removed and only labelled nanoparticles are remaining in the solution, this is followed by UV-visible spectrophotometry. The rinsing by ultrafiltration is continued until such time as no more change occurs in the intensity of this spectrum; in fact it diminishes when the free marker is removed, when there is no more of it and the spectrum remains unchanged in intensity, this signifies that there is no longer any marker other than the marker bound to the labelled nanoparticles. In addition, the fluorescent marker has a very specific absorption spectrum. In the case of the labelled nanoparticles, the absorption peak is at 580 nm, therefore slightly offset from that of the marker alone, which is at 585 nm, which thus demonstrates the formation of a bond between the label and the nanoparticle.

This labelling makes it possible to follow the nanoparticles in the cells by fluorescence microscopy such as confocal microscopy for example.

1. Nanoparticles of a metal chosen from among platinum, gold or bismuth, or one of the mixtures thereof, which are functionalised at their surface with functionalised polyethylene glycol.

2. A nanoparticle preparation method for preparing nanoparticles of a metal chosen from among platinum, gold or bismuth, or a mixture thereof, the method comprising:
a) mixing a precursor of nanoparticles with a functionalised polyethylene glycol comprising at least one OH, COOH, NH₂ or SH functional group, in water; and
b) exposing the mixture to ionizing radiation.

3. A method according to claim 2, wherein the ionizing radiation is chosen from gamma radiation deriving from a source of Cobalt 60 or Cesium 137, electrons or accelerated ions.

4. A method according to claim 2, i wherein the step of exposing extends over a period of time between 1 and 20 hours for a dose of 10 kGy applied with a source of gamma radiation from a Co⁶⁰ source having a dose rate of about 95.5 Gy/min in order to completely reduce 10⁻³ mol/L⁻¹ of platinum complex.

5. A method according to claim 2, wherein the functionalised polyethylene is chosen from among Hydroxyl poly(ethylene glycol) [PEG-OH], Poly(ethylene glycol) diamine [PEG-2NH₂], α-hydroxyl-ω-carboxyl poly(ethylene glycol) [HO-PEG-COOH], and PEG-thiol [PEG-SH].

6. A method according to claim 2, wherein the precursor is used in implementation in an amount ranging from 10⁻⁴ to 10⁻² mol/L, in terms of molar concentration of the total mixture of step a) and/or the functionalised polyethylene glycol is used in implementation in an amount ranging from 10⁻³ to 10⁻¹ mol/L, in terms of molar concentration of the total mixture of step a).

7. A method according to claim 2, wherein the molar ratio between the precursor and the functionalised polyethylene glycol is between 10 and 100.

8. A method according to claim 2, that is carried out in the absence of any solvent other than water.

9. (canceled)

10. (canceled)

11. The method according to claim 4, wherein the period of time is between 1 and 2 hours.

12. A nanoparticle according to claim 1, wherein the functionalised polyethylenes are chosen from among Hydroxyl poly(ethylene glycol) [PEG-OH], Poly(ethylene glycol) diamine [PEG-2NH₂], α-hydroxyl-ω-carboxyl poly(ethylene glycol) [HO-PEG-COOH], and PEG-thiol [PEG-SH].

13. Method for the treatment of cancers and tumours and/or for amplifying the effects of medical radiation used for the treatment of cancers or tumours, comprising administering to a patient in need thereof a therapeutically effective amount of a nanoparticle according to claim 1.

14. Method for the treatment of cancers and tumours and/or for amplifying the effects of medical radiation used for the treatment of cancers or tumours, comprising administering to a patient in need thereof a therapeutically effective amount of a nanoparticle obtainable by the method of claim 2.

15. Method for medical imaging for diagnostics in a subject in need thereof, comprising
administering the nanoparticle of claim 1 to the subject, and
performing the medical imaging on the subject.

16. Method for medical imaging for diagnostics in a subject in need thereof, comprising
administering to the subject a nanoparticle obtainable by the method of claim 2, and
performing the medical imaging on the subject.

17. The method of claim 15, wherein the medical imaging is performed by computed tomography (CT).

18. The method of claim 15, wherein the medical imaging is performed by computed tomography (CT).

19. The nanoparticles of claim 1, wherein the functionalised polyethylene glycol comprises at least one OH, COOH, NH₂ or SH functional group.
